SEQUENCE LISTING (10 sequences)

SEQ ID NO: 1 - DNA, 39 bp, Artificial
  synthetic oligonucleotide primer
ggggggggatccatgcccatggggtctctgcaaccgctg

SEQ ID NO: 2 - DNA, 45 bp, Artificial
  synthetic oligonucleotide primer
gggggcggccgcttattaggcgcgcttctcgcgctgcaccacgcg

SEQ ID NO: 3 - DNA, 71 bp, Artificial
  synthetic oligonucleotide primer
ataagaatgcggccgctaagtaagtaacttaagttccggttattttccacgatattgccgtcttttggcaa

SEQ ID NO: 4 - DNA, 42 bp, Artificial
  synthetic oligonucleotide primer
gccaaatacatggccatattatcatcgtgtttttcaaaggaa

SEQ ID NO: 5 - DNA, 582 bp, Artificial
  ctxA-S63K nucleotide sequence modified at nucleotides 187-189 (TCA to AAA)
aatgatgataagttatatcgggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccaccaaaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcg

SEQ ID NO: 6 - DNA, 31 bp, Artificial
  synthetic oligonucleotide primer
tgtttcccaccaaaattagtttgagaagtgc

SEQ ID NO: 7 - DNA, 30 bp, Artificial
  synthetic oligonucleotide primer
caaactaattttggtggaaacatatccatc

SEQ ID NO: 8 - DNA, 582 bp, Artificial
  ctxA1-E29H nucleotide sequence modified at nucleotides 85-87 (GAG to CAC)
aatgatgataagttatatcgggcagattctagacctcctgatgaaataaagcagtcaggtggtcttatgccaagaggacagagtcactactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcg

SEQ ID NO: 9 - DNA, 30 bp, Artificial
  synthetic oligonucleotide primer
caagaggacagagtcactactttgaccgag

SEQ ID NO: 10 - DNA, 30 bp, Artificial
  synthetic oligonucleotide primer
gttctcctgtctcagtgatgaaactggcac

(19) United States
(12) Patent Application Publication
(10) Pub. No.: US 2004/0171565 A1
(43) Pub. Date: Sep. 2, 2004

(54) DNA vaccines that expresses mutant ADP-ribosyltransferase toxins which display reduced, or are devoid of, ADP-ribosyltransferase activity

(76) Inventor: David Hone, Rockville, MD (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/632,095

(22) Filed: Aug. 1, 2003

Related U.S. Application Data
(60) Provisional application No. 60/447,460, filed on Feb. 14, 2003.

(51) Int. Cl. A61K 48/00

(52) U.S. Cl. 514/44; 435/320.1

(57) ABSTRACT

The present invention provides DNA vaccines that direct the coincident expression of vaccine antigens coincidently with mutant ADP-ribosyltransferase toxins (mARTs), which display reduced, or are devoid of, ADP-ribosyltransferase activity, and methods for vaccinating animals with the same. In particular, the present invention provides DNA vaccines that direct the coincident expression of vaccine antigens and mARTs that are useful for vaccinating against viral, bacterial, parasitic pathogens, autoimmune antigens and transplantation antigens.

This invention was made with the support of a grant from the National Institutes of Health (NIH) grant numbers R01-A14194 and R01-055367. The U.S. government has certain rights in this invention.

This application claims priority to U.S. Provisional Patent Application 60/447,460 filed on ______. The complete contents of that application are herein incorporated by reference.

### FIELD OF THE INVENTION

The present invention provides DNA vaccines that direct the coincident expression of vaccine antigens coincidently with mutant ADP-ribosyltransferase toxins (mARTs), which display reduced, or are devoid of, ADP-ribosyltransferase activity, and methods for vaccinating animals with the same. In particular, the present invention provides DNA vaccines that direct the coincident expression of vaccine antigens and mARTs that are useful for vaccinating against viral, bacterial, parasitic pathogens, autoimmune antigens and transplantation antigens.

### BACKGROUND OF THE INVENTION

I. DNA Vaccines:

DNA vaccines are defined in the present invention as DNA that is normally produced as a plasmid that can be introduced into animal tissue and therein expresses by animal cells to produce a messenger ribonucleic acid (mRNA) molecule, which is translated to produce one protein, one fragment of a protein or one fusion protein.

The prior art pertinent to the current invention describes a diverse array of conventional DNA vaccines, which are generally comprised of a plasmid vector, a promoter for transcription initiation that is active in eukaryotic cells, and a vaccine antigen (Gurunathan et al., Ann. Rev. Immunol., 18:927 (2000); Krieg, Biochim. Biophys. Acta., 1489:107 (1999); Cichutek, Dev. Biol. Stand., 100:119 (1999); Davis, Microbes Infect., 1:7 (1999); Leitner, Vaccine, 18:765 (1999)).

Examples of plasmid vectors that have been used in conventional DNA vaccines include pBR322 (ATCC# 31344); pUC19 (ATCC# 37254); pcDNA3.1 (Invitrogen, Carlsbad Calif. 92008; Cat. NO. V385-20; DNA sequence available at http://www.invitrogen.com/vectordata/index.html); pNGVL (National Gene Vector Laboratory, University of Michigan, Mich.); p414cyc (ATCC# 87380), p4l4GALS (ATCC# 87344), pBAD18 (ATCC# 87393), pBLCAT5 (ATCC# 77412), pBluescriptIIKS, (ATCC# 87047), pBSL130 (ATCC# 87145), pCM182 (ATCC# 87656), pCMVtkLUC (ATCC# 87633), pECV25 (ATCC#77187), pGEM-7zf (ATCC# 87048), pGEX-KN (ATCC# 77332), pJC20 (ATCC# 87113, pUB110 (ATCC# 37015), pUB18 (ATCC# 37253).

Examples of promoters that have been used in conventional DNA vaccines include the SV40 early promoter (Genebank accession # M99358, Fiers et al. Nature, 273: 113-120 (1978)), the cytomegalovirus immediate early promoter/enhancer (Genebank accession # AF025843) and the rous sarcoma virus long terminal repeat (Genebank accession # M83237; Lon et al. Hum. Immunol., 31: 229-235 (1991)) promoters, or the eukaryotic promoters or parts thereof, such as the β-casein (Genebank accession # AF194986; ref Fan et al. Direct submission (2000)), uteroglobin (Genebank accession # 10 NM003357; ref Hay et al. Am. J. Physiol., 268: 565-575 (1995)), β-actin (Genebank accession # NM001 101; ref Vandekerckhove and Weber. Proc. Natl. Acad. Sci. U.S.A., 73: 1106-1110 (1978)), ubiquitin (Genebank accession # AJ243268; Robinson. Direct Submission, (2000)) or tyrosinase (Genebank accession # NM000372; Shibaharo et al. Tohoku J. Exp. Med., 156: 403414 (1988)) promoters.

Examples of vaccine antigens that have been used in conventional DNA vaccines include Plasmodium vivax and Plasmodium falciparum antigens; Entamoeba histolytica antigens, Hepatitis C virus antigens, Hepatitis B virus antigens, HIV-1 antigens, Semliki Forest virus antigens, Herpes Simplex viral antigens, Pox virus antigens, Influenza virus antigens, Measles virus antigens, Dengue virus antigens, Papilloma virus antigens (A comprehensive reference database of DNA vaccine citations can be obtained from URL:-http://www.DNAvaccine.com/Biblio/articles.html). Since their inception in 1993, conventional DNA vaccines encoding an antigen under the control of a eukaryotic or viral promoters have been used to immunize rodents (e.g. mice, rats and guinea pigs), swine, chickens, ferrets, non-human primates and adult volunteers (Webster et al, Vacc., 12:1495-1498 (1994); Bernstein et al., Vaccine, 17:1964 (1999); Huang et al., Viral Immunol., 12:1 (1999); Tsukamoto et al., Virology, 257:352 (1999); Sakaguchi et al., Vaccine, 14:747 (1996); Kodihalli et al., J. Virol., 71: 3391 (1997); Donnelly et al., Vaccine, 15:865 (1997); Fuller et al., Vaccine, 15:924 (1997); Fuller et al., Immunol. Cell Biol., 75: 389 (1997); Le et al., Vaccine, 18:1893 (2000); Boyer et al., J. Infect. Dis., 181:476 (2000)).

II. Development of Adjuvants for Conventional DNA Vaccines:

Although conventional DNA vaccines induce immune responses against a diverse array of antigens, the magnitudes of the immune responses have not always been sufficient to engender protective immunity. Several approaches have been developed to increase the immunogenicity of conventional DNA vaccines, including the use of altered DNA sequences, such as the use of antigen-encoding DNA sequences optimized for expression in mammalian cells (Andre, J. Virol., 72:1497 (1998); Haas, et al., Curr. Biol. 6:315-24 (1996); zur Megede, et al., J. Virol., 74:2628 (2000); Vinner, et al., Vaccine, 17:2166 (1999)) or incorporation of bacterial immunostimulatory DNA sequence motifs (i.e. the CpG motif) (Krieg, Biochim. Biophys. Acta., 1489:107 (1999); McAdam et al. J. Virol., 74: 203-208 (2000); Davis, Curr. Top. Microbiol. Immunol., 247:17 (2000); McCluskie, Crit. Rev. Immunol., 19:303 (1999); Davis, Curr. Opin. Biotechnol., 8:635 (1997); Lobell, J. Immunol., 163:4754 (1999)). The immunogenicity of conventional DNA vaccines can also be modified by formulating the conventional DNA vaccine with an adjuvant, such as aluminum phosphate or aluminum hydroxyphosphate (Ulmer et al., Vaccine, 18:18 (2000)), monophosphoryl-lipid A (also referred to as MPL or MPLA; Schneerson et al. J. Immunol., 147: 2136-2140 (1991); Sasaki et al. Inf. Immunol., 65: 3520-3528 (1997); Lodmell et al. Vaccine, 18: 1059-1066 (2000)), QS-21 saponin (Sasaki, et al., J. Virol., 72:4931 (1998); dexamethasone (Malone, et al., J. Biol. Chem. 269:29903 (1994); CpG DNA sequences (Davis et al., J. Immunol., 15:870 (1998); lipopolysaccharide (LPS) antagonist (Shata and Hone, U.S. Patent Application (1999)), a cytokine (Hayashi et al. Vaccine, 18: 3097-3105 (2000); Sin et al. J. Immunol., 162: 2912-2921 (1999); Gabaglia et al. J. Immunol., 162: 753-760 (1999); Kim et al., Eur J Immunol., 28:1089 (1998); Kim et al., Eur. J. Immunol., 28:1089 (1998); Barouch et al., J. Immunol., 161:1875 (1998); Okada et al., J. Immunol., 159:3638 (1997); Kim et al., J. Virol., 74:3427 (2000)), or a chemokine (Boyer et al., Vaccine 17(Suppl 2):S53 (1999); Xin et al., Clin. Immunol., 92:90 (1999)). In each of the above cited instances the immunogenicity of the conventional DNA vaccines was enhanced or modified, thus validating the idea that the immunogenicity of conventional DNA vaccines can be influenced through the use of adjuvants.

III. Cholera Toxin is an Adjuvant

Cholera toxin (Ctx) is a well-known adjuvant that is typically used to augment the immunogenicity of mucosal vaccines, such as those given intranasally or orally (Xu-Amano, et al., J. Exp. Med., 178:1309 (1993); VanCott, et al., Vaccine, 14:392 (1996); Jackson, R. J. et al., Infect. Immun., 61:4272 (1993); Marinaro, M. et al., Ann. New York Acad. Sci., 795:361 (1996); Yamamoto, S. et al. J. Exp. Med. 185:1203 (1997); Porgador, et al., J. Immunol., 158:834 (1997); Lycke and Holmgren, Monogr., Allergy, 24:274 (1988); Homquist and Lycke, Eur. J. Immunol. 23:2136 (1993); Homquist, et al., Immunol., 87:220 (1996); Agren, et al., Immunol. Cell Biol., 76:280 (1998)). The adjuvant activity of Ctx is mediated by the Al domain of the A subunit of Ctx (herein referred to as CtxA1); chimeric proteins comprised of an antigen fused to CtxA1 demonstrate that CtxA1 alone possesses adjuvant activity (Agren, et al., J. Immunol., 164:6276 (2000); Agren, et al., Immunol. Cell Biol., 76:280 (1998); Agren, et al., J. Immunol., 158:3936 (1997)). The utilization of the A subunit, the Al domain of Ctx or analogues thereof in a DNA vaccine has not heretofore been reported. More recently the use of Ctx as an adjuvant has been extended to transcutaneous vaccines (Glenn et al., Infect. Immun., 67:1100 (1999); Scharton-Kersten et al., Vaccine 17(Suppl. 2):S37 (1999)). Thus, recent evidence suggests that cholera toxin (Ctx) as an adjuvant applied topically with an antigen to the skin surface (i.e. transcutaneous vaccination) elicits IgG responses against the antigen, whereas topical application of the antigen alone does not induce detectable IgG response (Glenn et al., sunrar (1999); Scharton-Kersten et al., so (1999)). Since Ctx is a member of the family of bacterial adenosine diphosphate-ribosylating exotoxins, other members of this family, E.g. the heat-labile toxins (Herein referred to as Ltx) of enterotoxigenic Escherichia coli, also possess adjuvant activity (Rappuoli et al., Immunol.

Today, 20:493 (1999)).

### SUMMARY OF THE INVENTION

The present invention describes novel compositions of DNA vaccines that express derivatives of ADP-ribosyltransferase toxins that display significantly reduced, or are deficient in, intrinsic ADP-ribosyltransferase activity (i.e. herein referred to as mARTs) and yet, as will be demonstrated below, retain adjuvanticity. DNA vaccines that express a mART are capable significantly augmenting immune responses to vaccine antigens encoded on DNA vaccines. Moreover, DNA vaccines that express a mART do not encumber the safety concern of DNA vaccines that express an active ADP-ribosyltransferase.

Heretofore, there is no documentation showing that mARTs, such as those derived from Ctx, heat labile toxin of enterotoxigenic Escherichia coli (Ltx ) or pertussis toxin (Ptx) and that display reduced or are devoid of ADP-ribosyltransferase activity are adjuvants in a DNA vaccine mode. That is, the present invention provides the first documentation demonstrating that DNA vaccines which direct the coexpression of a vaccine antigen and a mART are more effective than conventional DNA vaccines that express vaccine antigens alone. Moreover, DNA vaccines that direct the coincident expression of a vaccine antigen and a mART, which display reduced or is devoid of ADP-ribosyltransferase activity, are inherently safer than DNA vaccines that direct the coincident expression of a vaccine antigen and an active ADP-ribosyltransferase toxin.

Therefore, an object of the present invention is to provide DNA vaccines that express mARTs derived from Ctx.

Another object of the present invention is to provide DNA vaccines that express mARTs derived from Ltx or Ptx.

A further object of the present invention is to provide DNA vaccines that direct the coexpression of an antigen and a mART derived from Ctx.

A still further object of the present invention is to provide DNA vaccines that direct the coexpression of an antigen and a mART derived from Ltx or Ptx.

Yet another object of the invention is to provide DNA vaccines that express an antigen and said mARTs, and that can be used as prophylactic vaccines.

Still another object of the invention is to provide DNA vaccines that direct coexpression of an antigen, and said mARTs, and that can be used as therapeutic vaccines.

These and other objects of the present invention, which will be apparent from the detailed description of the invention provided hereinafter, have been met in one embodiment by providing DNA vaccines that direct the coincident expression of vaccine antigens and a mART and that induce potent immune responses to the vaccine antigen.

### DESCRIPTION OF THE DRAWING FIGURES

FIG. 1 shows the expression cassettes of various DNA vaccines configurations described in the Examples, wherein in each instance, the expression cassettes are located in expression vectors pcDNA3.1_{ZEO} or pRc/CMV, which place expression under the control of the CMV promoter (P_{CMV}).

FIG. 2 shows the expression cassettes of the DNA vaccines configurations that utilize two eukaryotic promoters (i.e., P₁ and P₂).

FIG. 3 is a graph showing the comparative results described in Example 5 where the serum IgG response against gp120 was significantly greater when the combination of a mART and antigen where used to vaccinate an animal than the IgG response obtained with a DNA vaccine that expressed gp120 alone.

FIG. 4 is a schematic showing intracellular trafficking pathways employed by purified holotoxin, compared to CtxA1-S63K when expressed by a DNA vaccine.

FIG. 5 is a schematic of an intracellular trafficking pathway showing that delivery of mARTs by the DNA vaccine mode bypasses the golgi apparatus.

FIG. 6 is a schematic of an intracellular trafficking pathway showing increased membrane recycling and maturation of dendritic cells that harbor DNA vaccine into a mature antigen presenting cell.

### DETAILED DESCRIPTION OF THE INVENTION

I. Generic Structure of DNA Vaccines that Direct the Coincident Expression of a Vaccine Antigen and a mART:

The particular novel DNA vaccines, which direct the coincident expression of a vaccine antigen and a mART, employed in the present invention are engineered using one of the three following preferable configurations.

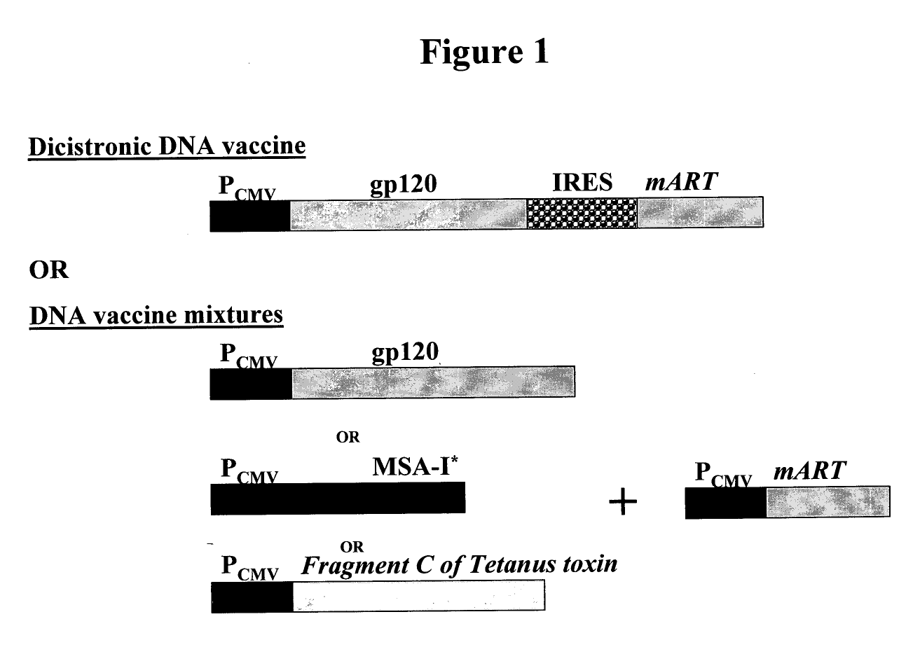

1. In one configuration, the DNA vaccine that expresses an antigen and a mART is composed of an expression vector, one eukaryotic promoter, a mART and at least one vaccine antigen, wherein the mART and the vaccine antigen are separated by a eukaryote internal ribosome entry site (herein referred to as an “IRES”; see FIG. 1).

2. In the second configuration of DNA vaccines that express a mART is composed of an expression vector, a eukaryotic promoter, and a mART. A diagrammatic depiction of this generic mART DNA vaccine configuration is shown in FIG. 1. Prior to vaccination, mART DNA vaccines of this configuration are mixed with a DNA vaccine that expresses a vaccine antigen.

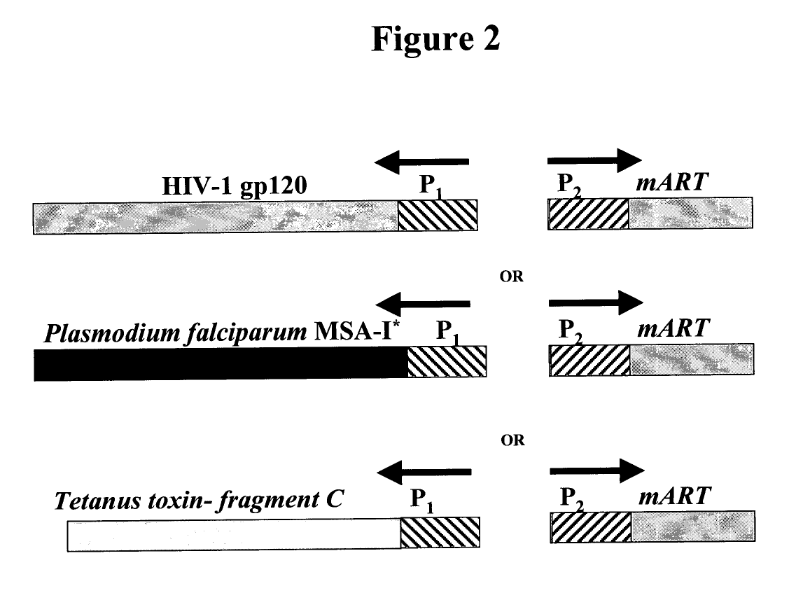

3. In the third configuration, the DNA vaccine that expresses an antigen and a mART is composed of an expression vector, two eukaryotic promoters, a mART and at least one vaccine antigen. A diagrammatic depiction of a generic DNA vaccine that expresses mART and an immunogen using two eukaryotic promoters is shown in FIG. 2.

The particular mART is not critical to the present invention and may be derived from the A subunit of cholera toxin (i.e. CtxA; GenBank accession no. X00171, AF175708, D30053, D30052,), or parts thereof (i.e. the A1 domain of the A subunit of Ctx (i.e. CtxA1; GenBank accession no. K02679)), from any classical Vibrio cholerae (E.g. V. cholerae strain 395, ATCC # 39541) or E1 Tor V. cholerae (E.g. V. cholerae strain 2125, ATCC # 39050) by introducing mutations including but not restricted to replacement of arginine-7 with lysine (herein referred to as “R7K”), glutamine-29 with histidine (E29H), leucine-41 with phenylalanine (L41F), serine-61 with lysine (S61K), serine-63 with lysine (S63K), serine-63 with tyrosine (S63Y), valine-53 with aspartic acid (V53D), valine-97 with lysine (V97K), tyrosine-104 with lysine (Y104K), proline-106 with serine (P106S), histidine-171 with tyrosine (H171Y), or combinations thereof. Such mutants are made by conventional site-directed mutagenesis procedures, as described below.

Alternatively, the mART may be derived from the A subunit of heat-labile toxin (referred to herein as “LtxA” of enterotoxigenic Escherichia coli (GenBank accession # M35581) isolated from any enterotoxigenic Escherichia coli, including but not restricted to E. coli strain H10407 (ATCC # 35401), by introducing mutations including but not restricted to R7K, E29H, L41F, S61K, S63K, V53D, V97K, P106S Y104K, H171Y, or combinations thereof. Such mutants are made by conventional site-directed mutagenesis procedures, as described below.

Yet another alternative, the particular mART is not critical to the present invention and may be derived from pertussis toxin (i.e. Ptx), or parts thereof (i.e. the A subunit of Ptx (i.e. PtxA), wherein said ptx gene can be isolated from Bordetella, such as but not restricted to Bordetella pertussis (i.e. ATCC No. 10380; GenBank accession no. M13223), B. bronchiseptica (ATCC No. 10580; GenBank accession no. M16492) or B. parapertussis (ATCC No. 15237; GenBank accession no. M16493), by introducing mutations including but not restricted to mutations that replace arginine-9 with serine (i.e. “R9S”), arginine-13 with histidine (i.e. R13H), histidine-35 with arginine (i.e. H35R) or phenylalanine-50 with serine (i.e. F50S), or combinations thereof. Such mutants are made by conventional site-directed mutagenesis procedures, as described below.

Mutations that reduce or eliminate the catalytic activity of the target ADP-ribosyltransferase toxin (e.g. CtxA, LtxA or PtxA) can be introduced into gram-negative bacteria using any well-known mutagenesis technique. These include but are not restricted to: (a) non-specific mutagenesis, using chemical agents such as N-methyl-N′-nitro-N-nitrosoguanidine, acridine orange, ethidium bromide, or non-lethal exposure to ultraviolet light (Miller (Ed), 1991, In: A short course in bacterial genetics, Cold Spring Harbor Press, Cold Spring Harbor, N.Y.); (b) Site-Directed mutagenesis by conventional procedures (Miller, 1991, supra) or using QuikChange® Site-Directed Kit (Catalog #200518, Stratagene). The latter site-directed mutagenesis process entails whole-plasmid PCR using the target plasmid (e.g. pOGL1-A1) as template, and forward and reverse primers that modify the target nucleotides (e.g. replace nucleotides 187-189 in CtxA1 (i.e. the serine-63 TCA codon) with a lysine codon (i.e. 5′-AAA); See Examples). The PCR-generated plasmids are digested with DpnI to remove the template DNA and the digested DNA was introduced into E. coli Stable2® by standard transformation procedures (Miller, 1991, supra). The transformed bacilli are cultured at 30° C. for 16 hr on solid media (e.g. tryptic soy agar; Difco, Detroit Mich.) supplemented with the appropriate antibiotic corresponding to the antibiotic-resistance gene on the target plasmid (e.g. 100-μg/ml ampicillin).

Isolated colonies that grow on the solid media are selected and grown overnight in 3 ml of liquid media (e.g. Luria-Bertani broth, Difco) supplemented with the appropriate antibiotic corresponding to the antibiotic-resistance gene on the target plasmid (e.g. 100-μg/ml ampicillin). Supercoiled plasmid DNA is extracted from the overnight liquid cultures using a Qiagen® Mini Plasmid DNA Preparation Kit (Cat No Q7106). To screen for an appropriate mutant derivative, plasmid preparations are subjected to PCR using primers specific for the mART allele and the PCR-generated products are analyzed by agarose gel electrophoresis. Clones carrying plasmids that prove positive for mART allele are stored at −80° C. and used as the source of DNA for the vaccination studies.

Standard procedures are used to construct each mART allele, including those in constant use in our laboratory ([1-11]; App. 2,3). Typically, the DNA sequence of each component of a proposed DNA vaccine is downloaded and a plasmid construction strategy is generated using Clone Manager® software version 4.1 (Scientific and Educational Software Inc., Durhan N.C.). This software enables the design PCR primers and the selection of restriction endonuclease (RE) sites that are compatible with the specific DNA fragments being manipulated.

REs (New England Biolabs Beverly, Mass.), T4 DNA ligase (New England Biolabs, Beverly, Mass.) and Taq polymerase (Life technologies, Gaithersburg, Md.) are used according to the manufacturers' protocols; Plasmid DNA is prepared using small-scale (Qiagen Miniprep^{R} kit, Santa Clarita, Calif.) or large-scale (Qiagen Maxiprep^{R} kit, Santa Clarita, Calif.) plasmids DNA purification kits according to the manufacturer's protocols (Qiagen, Santa Clarita, Calif.); Nuclease-free, molecular biology grade milli-Q water, Tris-HCl (pH 7.5), EDTA pH 8.0, 1M MgCl₂, 100% (v/v) ethanol, ultra-pure agarose, and agarose gel electrophoresis buffer may be purchased from Life Technologies (Gaithersburg, Md.). DNA ligation reactions and agarose gel electrophoresis are conducted according to well-known procedures (Sambrook, et al., supra (1989); (Ausubel, et al., supra (1990)).

PCRs are conducted in a Strategene Robocycler, model 400880 (Strategene). Primer annealing, elongation and denaturation times in the PCRs may be set according procedures online in our laboratory (App. 2,3). E. coli strain Sable2^{R} (LifeTechnologies) can serve as the initial host of each new recombinant plasmid. DNA is introduced into E. coli Stable2® by standard transformation procedures (Sambrook, et al., supra (1989); (Ausubel, et al., supra (1990)).

Transformed Stable2® bacilli are cultured at 30° C. for 16 hr on solid agar (e.g. tryptic soy agar; Difco, Detroit Mich.) supplemented with the appropriate antibiotic corresponding to the antibiotic-resistance gene on the target plasmid (e.g. 100-μg/ml ampicillin). Isolated colonies that grow on the solid media are selected and grown overnight in 3 to 10 ml of liquid media (e.g. Luria-Bertani broth, Difco) supplemented with the appropriate antibiotic corresponding to the antibiotic-resistance gene on the target plasmid (e.g. 100-μg/ml ampicillin). Supercoiled plasmid DNA is extracted from the overnight liquid cultures using a Qiagen® Mini Plasmid DNA Preparation Kit (Cat No Q7106).

To screen for an appropriate allelic or mutant derivative, plasmid and chromosomal DNA preparations are subjected to PCR using primers specific for the target allele and the PCR-generated products are analyzed by agarose gel electrophoresis. Clones carrying the appropriate alleles and plasmids are stored at −80° C. Dideoxynucleotide sequencing may also be conducted to verify that the appropriate nucleotides were introduced into the target Salmonella strains, using conventional automated DNA sequencing techniques [12] and an Applied Biosystems automated sequencer, model 373A (Foster City, Calif.).

The expression of immunogens by the modified recombinant DNA vaccines is confirmed by introducing each plasmid into mammalian cells (e.g. Chinese Hamster Ovary cells; ATCC # CCL-61) using standard transfection procedures (Sambrook, et al., supra (1989); (Ausubel, et al., supra (1990)) and a commercially available transfection kit (e.g. the FuGENE^{R} Transfection System; Roche Molecular Biochemicals, Indianapolis, Ind.). Lysates of the transfected cells and culture supernatants are prepared after incubating 72 hr at 370C in 5% CO2, and are fractionated by SDS-polyacrylamide gel electrophoresis and transferred to nitrocellulose filter [7]. Following transfer, the immunogen may be detected on the filter using a standard immunochemical procedure and mAbs specific for the immunogen as primary antibodies, as described by our group previously [7]. Similarly, plasmids that carry wild type, synthetic or mutant ADP-ribosyltransferase alleles (e.g. CtxA1-S63K) may be assessed for ADP-ribosylation activity by transiently transfecting mammalian cells (e.g. Chinese Hamster Ovary cells; ATCC # CCL-61) as above and determined the level of cAMP production by transfected cells using a quantitative cAMP colorimetric assay (Amersham, San Francisco, Calif.), as per the manufacture's instructions.

Selection of the appropriate methodology will depend on the target and will be obvious to those skilled in the art.

II. Expression Vectors Useful for DNA Vaccines that Express a mARTs:

The particular expression vector employed in the present invention is not critical thereto, and can be selected from any of the commercially available expression vectors, such as pcDNA3.1 ZEO (Invitrogen Cat.# V790-20), pRc/CMV (Genebank accession E14286) obtained from Invitrogen Corporation (San Diego, Calif.); pNGVL (National Gene Vector Laboratory, University of Michigan, Mich.); pXT1 (Genebank accession M26398)or pSG5 (Genebank accession Af0113258), obtained from Stratagene (La Jolla, Calif.); pPUR (Genebank accession U07648) or pMAM (Genebank accession U02443) obtained from ClonTech (Palo Alto, Calif.); pDual (Genbank accession # AF041247); pG51uc (Genbank accession # AF264724); pACT (Genbank accession # AF264723); pBIND (Genbank accession # AF264722); pCI-Neo (Genbank accession # U47120); pCMV-BD (Genbank accession # AF151088); pIRES-P (Genbank accession # Z75185); pRL-CMV (Genbank accession # AF025843), or by adaptation of a publicly or commercially available eukaryotic expression system.

III. Promoter, useful for DNA Vaccines that Express mARTs:

The particular promoter employed in the present invention is not critical thereto, and can be selected from promoters well-known to be useful for driving expression of genes in animal cells, such as the viral promoters or parts or derivatives thereof, such as the cytomegalovirus immediate early promoter/enhancer (Genebank accession # AF025843) and rous sarcoma virus long terminal repeat (Genebank accession # M83237; Lon et al. Hum. Immunol., 31: 229-235 (1991)) promoters.

Alternatively, the promoter employed in the present invention can be selected from eukaryotic promoters useful for driving expression of genes in animal cells or parts thereof, including but not restricted to the p-casein promoter (Genebank accession # AF 194986; Fan et al. Direct submission (2000)), uteroglobin promoter (Genebank accession # NM003357; Hay et al. Am. J. Physiol., 268: 565-575 (1995)), the desmin gene promoter that is only active in muscle cells (Loirat et al., Virology, 260:74 (1999));the constitutively expressed p-actin promoter (Genebank accession # NM001101; Vandekerckhove and Weber. Proc.

Natl. Acad. Sci. U.S.A., 73: 1106-1110 (1978)), ubiquitin (Genebank accession # AJ243268) or the tyrosinase promoter (Genebank accession # NM000372; Shibaharo et al. J. Exp. Med., 156: 403-414 (1988)).

Although the particular promoter is not critical to the present, there may be exceptions when the object is to selectively target expression to specific cell types. In this case, the selected promoter is one that is only active in the target cell type. Examples of tissue specific promoters include, but are not limited to, S1- and β-casein promoters which are specific for mammary tissue (Platenburg et al, Trans. Res., 3:99-108 (1994); and Maga et al, Trans. Res., 3:36-42 (1994)); the phosphoenolpyruvate carboxykinase promoter which is active in liver, kidney, adipose, jejunum and mammary tissue (McGrane et al, J. Reprod. Fert., 41:17-23 (1990)); the tyrosinase promoter which is active in lung and spleen cells, but not testes, brain, heart, liver or kidney (Vile et al, Canc. Res., 54:6228-6234 (1994)); the involucerin promoter which is only active in differentiating keratinocytes of the squamous epithelia (Carroll et al, J. Cell Sci., 103:925-930 (1992)); the uteroglobin promoter which is active in lung and endometrium (Helftenbein et al, Annal. N. Y. Acad. Sci., 622:69-79 (1991)); the desmin gene promoter that is only active in muscle cells (Loirat et al., Virology, 260:74 (1999)).

Genetic engineering procedures and reagents for the preparation of the promoters described in this section are detailed below.

IV. Internal Ribosome Entry Sites useful for Bicistronic DNA Vaccines that Express mARTs:

Translation of mRNA in eukaryotic cells requires the presence of a ribosomal recognition signal. Prior to initiation of translation of mRNA in eukaryotic cells, the 5-prime end of the mRNA molecule is “capped” by addition of methylated guanylate to the first mRNA nucleotide residue (Lewin, Genes V, Oxford University Press, Oxford (1994); Darnell et al, Molecular Cell Biology, Scientific American Books, Inc., W.H. Freeman and Co., New York, N.Y. (1990)). It has been proposed that recognition of the translational start site in mRNA by the eukaryotic ribosomes involves recognition of the cap, followed by binding to specific sequences surrounding the initiation codon on the mRNA. After recognition of the mRNA by the ribosome, translation initiates and typically produces a single protein species per mRNA molecule (Lewin, Genes V, Oxford University Press, Oxford (1994); Darnell et al, Molecular Cell Biology, Scientific American Books, Inc., W.H. Freeman and Co., New York, N.Y. (1990)).

It is possible for cap independent translation initiation to occur and/or to place multiple eukaryotic coding sequences within a eukaryotic expression cassette if an internal ribosome entry sequence (IRES) is present on the mRNA molecule (Duke et al, J. Virol., 66:1602-1609 (1992)). IRES are used by viruses and occasionally in mammalian cells to produce more than one protein species per mRNA molecule as an alternative strategy to mRNA splicing ((Creancier, et al., J. Cell. Biol., 150:275 (2000); Izquierdo and Cuezva, Biochem. J., 346:849 (2000)).

The particular IRES employed in the present invention is not critical and can be selected from any of the commercially available vectors that contain IRES sequences such as those located on plasmids pCITE4a-c (Novagen, URL:-http//www.novagen.com; U.S. Pat. No. 4,937,190); pSLIRES11 (Accession: AF171227; pPV (Accession # Y07702); pSVIRES-N (Accession #: AJ000156); Creancier et al. J. Cell Biol., 10: 275-281 (2000); Ramos and Martinez-Sala, RNA, 10: 1374-1383 (1999); Morgan et al. Nucleic Acids Res., 20: 1293-1299 (1992); Tsukiyama-Kohara et al. J. Virol., 66: 1476-1483 (1992); Jang and Wimmer et al. Genes Dev., 4: 1560-1572 (1990)), or on the Bicistronic retroviral vector (Accession #: D88622); or found in eukaryotic cells such as the Fibroblast growth factor 2 IRES for stringent tissue-specific regulation (Creancier, et al., J. Cell. Biol., 150:275 (2000)) or the Internal-ribosome-entry-site of the 3′-untranslated region of the mRNA for the beta subunit of mitochondrial H⁺-ATP synthase (Izquierdo and Cuezva, Biochem. J., 346:849 (2000)).

Genetic engineering procedures and reagents for the preparation of the IRES described in this section are detailed below.

V. Antigens useful for DNA Vaccines that Direct the Coincident Expression of Antigens and mARTs:

The novel DNA vaccines of the present invention encode antigens that may be either foreign antigens or endogenous antigens.

As used herein, “foreign antigen” refers to a protein or fragment thereof, which is foreign to the recipient animal cell or tissue, such as, but not limited to, a viral protein, a parasite protein, an immunoregulatory agent, or a therapeutic agent.

An “endogenous antigen” refers to a protein or part thereof that is naturally present in the recipient animal cell or tissue, such as, but not limited to, a cellular protein, a immunoregulatory agent, or a therapeutic agent.

The foreign antigen may be a protein, an antigenic fragment or antigenic fragments thereof that originate from viral and parasitic pathogens.

Alternatively, the foreign antigen may be encoded by a synthetic gene and may be constructed using conventional recombinant DNA methods (See example 1 for synthetic gene construction procedures); the synthetic gene may express antigens or parts thereof that originate from viral and parasitic pathogens. These pathogens can be infectious in humans, domestic animals or wild animal hosts.

The foreign antigen can be any molecule that is expressed by any viral, bacterial or parasitic pathogen prior to or during entry into, colonization of, or replication in their animal host.

The viral pathogens, from which the viral antigens are derived, include, but are not limited to, Orthomyxoviruses, such as influenza virus (Taxonomy ID: 59771; Retroviruses, such as RSV, HTLV-1 (Taxonomy ID: 39015), and HTLV-II (Taxonomy ID: 11909), Herpesviruses such as EBV Taxonomy ID: 10295); CMV (Taxonomy ID: 10358) or herpes simplex virus (ATCC #: VR-1487); Lentiviruses, such as HIV-1 (Taxonomy ID: 12721) and HIV-2 Taxonomy ID: 11709); Rhabdoviruses, such as rabies; Picomoviruses, such as Poliovirus (Taxonomy ID: 12080); Poxviruses, such as vaccinia (Taxonomy ID: 10245); Rotavirus (Taxonomy ID: 10912); and Parvoviruses, such as adeno-associated virus 1 (Taxonomy ID: 85106).

Examples of viral antigens can be found in the group including but not limited to the human immunodeficiency virus antigens Nef (National Institute of Allergy and Infectious Disease HIV Repository Cat. # 183; Genbank accession # AF238278), Gag, Env (National Institute of Allergy and Infectious Disease H1V Repository Cat. # 2433; Genbank accession # U39362), Tat (National Institute of Allergy and Infectious Disease HIV Repository Cat. # 827; Genbank accession # M13137), mutant derivatives of Tat, such as Tat-A3145 (Agwale et al. Proc. Natl. Acad. Sci. In press Jul 8^{th} (2002)), Rev (National Institute of Allergy and Infectious Disease HWV Repository Cat. # 2088; Genbank accession # L14572), and Pol (National Institute of Allergy and Infectious Disease HIV Repository Cat. # 238; Genbank accession # AJ237568) and T and B cell epitopes of gpl20 (Hanke and McMichael, AIDS Immunol Lett., 66:177 (1999); Hanke, et al., Vacine, 17:-89 (1999); Palker et al, J. Immunol, 142-361 2-361 9 (1989)) chimeric derivatives of HWV-1 Env and gp120, such as but not restricted to fusion between gp120 and CD4 (Fouts et al., J. Virol. 2000, 74.11427-11416 (2000)); truncated or modified derivatives of HIV-1 env, such as but not restricted to gp140 (Stamatos et al. J. Virol, 72-9656-9667 (1998)) or derivatives of HIV-1 Env and/or gp140 thereof (Binley, et al. J. Virol, 76-2606-261 6 (2002); Sanders, et al. J. Virol, 74:5091 5100 (2000); Binley, et al. J. Virol, 74627-643 (2000)), the hepatitis B surface antigen (Genbank accession # AF043578; Wu et al, Proc. Natl. Acad. Sci., USA, 6:4726-4730 (1989)); rotavirus antigens, such as VP4 (Genbank accession # AJ293721; Mackow et al, Proc. Natl. Acad. Sci., USA, 87:518-522 (1990)) and VP7 (GenBank accession # AY003871; Green et al, J. Virol., 62:1819-1823 (1988)), influenza virus antigens such as hemagglutinin or (GenBank accession # AJ404627; Pertmer and Robinson, Virology, 257:406 (1999)); nucleoprotein (GenBank accession # AJ289872; Lin et al, Proc. Natl. Acad. Sci., 97: 9654-9658 (2000)) ) herpes simplex virus antigens such as thymidine kinase (Genbank accession # AB047378; Whitley et al, In: New Generation Vaccines, pages 825-854).

The bacterial pathogens, from which the bacterial antigens are derived, include but are not limited to, Mycobacterium spp., Helicobacter pylori, Salmonella spp., Shigella spp., E. coli, Rickettsia spp., Listeria spp., Legionella pneumoniae, Pseudomonas spp., Vibrio spp., and Borellia burgdorferi.

Examples of protective antigens of bacterial pathogens include the somatic antigens of enterotoxigenic E. coli, such as the CFA/I fimbrial antigen (Yamamoto et al, Infect. Immun., 50:925-928 (1985)) and the nontoxic B-subunit of the heat-labile toxin (Klipstein et al, Infect. Immun., 4.0:888-893 (1983)); pertactin of Bordetella pertussis (Roberts et al, Vacc., 1(:43-48 (1992)), adenylate cyclase-hemolysin of B. pertussis (Guiso et al, Micro. Path., 11:423-431 (1991)), fragment C of tetanus toxin of Clostridium tetani (Fairweatheretal, Infect. Immun., 5:1323-1326 (1990)), OspA of Borellia burgdorferi (Sikand, et al. Pediatric, 108:123-128 (2001); Wallich, et al. Infect Immun, 69-2130)-2116 (2001)), protective paracrystalline-surface-layer proteins of Rickettsia prowazekii and Rickettsia typhi (Carl, et al. Proc Nqtl Acad Sci U S A, 87-9237-8241 (1990)), the listeriolysin (also known as “Llo” and “Hly”) and/or the superoxide dismutase (also know as “SOD” and “p60”) of Listeria monocytogenes (Hess, J., et al. Infect Immun. 65-1286-92 (1997); Hess, J., et al. Proc. Natl. Acad. Sci. 93-1459-1463 (1996); Bouwer, et al. J. Exp. Med. 175-1467-71 (1992)), the urease of Helicobacter pylori (Gomez-Duarte, et al. Vaccine 16 460-71 (1998); Corthesy-Theulaz, et al. Infection & Immunity 66, 581-6 (1998)), and the receptor-binding domain of lethal toxin and/or the protective antigen of Bacillus anthrax (Price, et al. Infect. Immun. 69, 4509-4515 (2001)).

The parasitic pathogens, from which the parasitic antigens are derived, include but are not limited to, Plasmodium spp.,such as Plasmodium falciparum (ATCC#: 30145); Trypanosome spp.,such as Trypanosoma cruzi (ATCC#: 50797); Giardia spp., such as Giardia intestinalis (ATCC#: 30888D); Boophilus spp., Babesia spp.,such as Babesia microti (ATCC#: 30221); Entamoeba spp.,such as Entamoeba histolytica (ATCC#: 30015); Eimeria spp.,such as Eimeria maxima (ATCC# 40357); Leishmania spp.,(Taxonomy ID: 38568); Schistosome spp., such as Schistosoma mansoni (Genbank accession # AZ301495) Brugia spp., such as Brugia malayi (Genbank accession # BE352806) Fascida spp., such as Fasciola hepatica (Genbank accession # AF286903) Dirofilaria spp., such as Dirofilaria immitis (Genbank accession # AF008300) Wuchereria spp., such as Wuchereria bancrofti (Genbank accession # AF250996) and Onchocerea spp; such as Onchocerca volvulus (Genbank accession # BE588251).

Examples of parasite antigens can be found in the group including but not limited to the pre-erythrocytic stage antigens of Plasmodium spp. (Sadoff et al, Science, 24:336-337 (1988); Gonzalez, et al., J. Infect. Dis., 169:927 (1994); Sedegah, et al., Proc. Natl. Acad. Sci. 91:9866 (1994); Gramzinski, et al., Vaccine, 15:913 (1997); Hoffinan, et al., Vaccine, 15:842 (1997)) such as the circumsporozoite antigen of P. falciparum (GenBank accession # M22982) or P vivax (GenBank accession # M20670); the liver stage antigens of Plasmodium spp. (Hollingdale et al., Ann. Trop. Med Parasitol., 92:411 (1998), such as the liver stage antigen 1 (as referred to as LSA-1; GenBank accession # AF086802); the merozoite stage antigens of Plasmodium spp. (Holder et al., Parassitologia, 41:409 (1999); Renia et al., Infect. Immun., 65:4419 (1997); Spetzler et al, Int. J. Pept. Prot. Res., 43:351-358 (1994)), such as the merozoite surface antigen-i (also referred to as MSA-1 or MSP-1; GenBank accession # AF199410); the surface antigens of Entamoeba histolytica (Mann et al, Proc. Natl. Acad. Sci., USA, 88:3248-3252 (1991)), such as the galactose specific lectin (GenBank accession # M59850) or the serine rich Entamoeba histolytica protein (also referred to as SREHP; Zhang and Stanley, Vaccine, 18:868 (1999)); the surface proteins of Leishmania spp. (also referred to as gp63; Russell et al, J. Immunol., 1(:1274-1278 (1988); Xu and Liew, Immunol., 84: 173-176 (1995)), such as 63 kDa glycoprotein (gp63) of Leishmania major (GenBank accession # Y00647 or the 46 kDa glycoprotein (gIA6) of Leishmania major (Handman et al, Vaccine, 18: 3011-3017 (2000); paramyosin of Brugia malayi (GenBank accession # U77590; Li et al, Mol. Biochem. Parasitol., 49:315-323 (1991)), the triose-phosphate isomerase of Schistosoma mansoni (GenBank accession # W06781; Shoemaker et al, Proc. Natl. Acad. Sci., USA, 89:1842-1846 (1992)); the secreted globin-like protein of Trichostrongylus colubriformis (GenBank accession # M63263; Frenkel et al, Mol. Biochem. Parasitol., 5(1:27-36 (1992)); the glutathione-S-transferase's of Fasciola hepatica (GenBank accession # M77682; Hillyer et al, Exp. Parasitol., 75:176-186 (1992)), Schistosoma bovis (Genbank accession # M77682) and S. japonicum (GenBank accession # U58012; Bashir et al, Trop. Geog. Med., 46:255-258 (1994)); and KLH of Schistosoma bovis and S. japonicum (Bashir et al, supra).

As mentioned earlier, DNA vaccine formulations that direct the coexpression of an antigen and mARTs may encode an endogenous antigen, which may be any cellular protein, cytokine, chemokine, or parts thereof, that may be expressed in the recipient cell, including but not limited to tumor antigens, or fragments and/or derivatives of tumor antigens, thereof. Thus, in the present invention, a DNA vaccine that co-expresses an antigen and a mART may encode a tumor antigen or parts or derivatives thereof. Alternatively, DNA vaccines that co-express an antigen and a mART may encode synthetic genes, which encode tumor-specific antigens or parts thereof.

Examples of tumor specific antigens include prostate specific antigen (Gattuso et al, Human Pathol., 26:123-126 (1995)), TAG-72 and CEA (Guadagni et al, Int. J. Biol. Markers, 9:53-60 (1994)), human tyrosinase (GenBank accession # M27160; Drexler et al., Cancer Res., 59:4955 (1999); Coulie et al, J. Immunothera., 14:104-109 (1993)), tyrosinase-related protein (also referred to as TRP; GenBank accession # AJ132933; Xiang et al., Proc. Natl. Acad. Sci., 97:5492 (2000)); tumor-specific peptide antigens (Dyall et al., J. Exp. Med., 188:1553 (1998).

Genetic engineering procedures and reagents for the preparation of the novel DNA vaccines described in this section are detailed below.

VI Genetic Engineering Procedures

The novel DNA vaccines described herein are produced using procedures well known in the art, including polymerase chain reaction (PCR; Sambrook, et al., Molecular cloning; A laboratory Manual: Vol. 1-3, Cold Spring Harbor Laboratory Press, Cold Spring Harbor, N.Y. (1989)); DNA synthesis using an Applied Biosystems DNA synthesizer (Perkin Elmer ABI 3948, using the standard cycle as according to procedures provided by the manufacturer); agarose gel electrophoresis (Ausubel, Brent, Kingston, Moore, Seidman, Smith and Struhl. Current Protocols in Molecular Biology: Vol. 1 and 2, Greene Publishing Associates and Wiley-Interscience, New York (1990)); restriction endonuclease digestion of DNA (Sambrook, et al., vupra (1989)); annealing DNA fragments using bacteriophage T4 DNA ligase (New England Biolabs, Cat #202CL; Sambrook, Fritsch, and Maniatis. Molecular cloning; A laboratory Manual: Vol. 1-3, Cold Spring Harbor Laboratory Press, Cold Spring Harbor, N.Y. 1989)); introducing recombinant plasmids into Escherichia coli by electrotransformation (also called electroporation; (Sambrook, et al., Tunr, (1989)); culturing of E. coli isolates that carry recombinant plasmids on solid media (e.g. Tryptic Soy Agar; Beckton Dickenson, Sparks, Md. cat #211046) or in liquid media (e.g. Tryptic Soy Broth; Beckton Dickenson, Sparks, Md. cat #211771) containing the appropriate antibiotics (e.g. 100 μg/ml ampicillin 20 μg/ml chloramphenicol or 50 μg/ml kanamycin) for the selection of bacteria that carry the recombinant plasmid; isolation of plasmid DNA using commercially available DNA purification kits (Qiagen, Santa Clarita, Calif. EndoFree Plasmid Maxi Kit, cat # 12362); transfection of murine and human cells using the FuGENE^{R} proprietary multi-component transfection system using the procedure recommended by the manufacturer (Roche Diagnostics Corporation, Roche Molecular Biochemicals, Indianapolis, Ind. cat # 1 815 091; e.g. Schoonbroodt and Piette, Biochemica 1:25 (1999)); culturing murine or human cells lines in RPMI 1640 medium (Life Technologies, Gaithersburg Md.) containing 10% (v/v) fetal calf serum (Gemini Bioproducts, Calabasas, Calif. cat #100-107; See also Current Protocols in immunology, Greene Publishing Associates and Wiley-Interscience, New York (1990)); analysis of tissue culture supernatants and cell lysates by sodium dodecylsufate-polyacrylamide gel electrophoresis (SDS-PAGE; Harlow and Lane. Using Antibodies, A Laboratory Manual, Cold Spring Harbor Laboratory Press, NY, (1988)) and immunoblotting (Harlow and Lane. Using Antibodies, A Laboratory Manual. Cold Spring Harbor Laboratory Press, NY, (1988)); quantitation of recombinant proteins produced by recombinant plasmids in murine or human cells using a semi-quantitative immunoblot (Abacioglu, Y. H. et al., AIDS Res. Hum. Retroviruses 10:371 (1994)), or a capture enzyme-linked immunosorbent assay (ELISA; Ausubel, et al., Current Protocols in Molecular Biology: Vol. 1 and 2, Greene Publishing Associates and Wiley-Interscience, New York (1990)); quantitative reverse trascriptase (RT)-PCR is conducted as described (Ausubel, et al., In: Current Protocols in Molecular Biology: Vol. 1 and 2, Greene Publishing Associates and Wiley-Interscience, New York (1990)), using the Thermoscript RT-PCR System according to the manufacturer's directions (Life Technologies, Gaithersburg MD; cat #11146-016).

VTI Generation of Specific DNA Sequences:

DNA sequences encoding the individual components of the novel DNA vaccines of the present invention, such as the promoter/enhancer, antigen, internal ribosome entry site (IRESs), and the mART may be obtained from the American Type Culture Collection (ATCC, Manassas, Va.). Recombinant bacteria containing the plasmids that encode the genes of interest are cultured as described above; the plasmid DNA is purified and the target sequence is isolated and analyzed by restriction endonuclease digestion or by PCR (Protocols for these procedures are provided above).

Alternatively, in instances where the desired DNA sequence is not available at the ATCC, individual DNA sequences can be made de novo using a DNA sequence obtained from GenBank or from commercial gene databases, e.g. Human Genome Sciences (Gaithersburg, Md.), as the blueprint of the target gene, DNA fragment, or parts thereof. Thus, de novo-generated DNA encoding promoter/enhancers, antigens, internal ribosome entry sites (IRESs), and mARTs are synthesized using procedures well known in the art (Andre et al., supra, (1998); et al., Haas supra, (1996)). Briefly, the procedure entails a step-by-step approach, wherein synthetic oligonucleotides 100-200 nucleotides in length (i.e. preferably with sequences at the 5′- and 3′ ends that match at the 5′ and 3′ ends of the oligonucleotides that encodes the adjacent sequence) are produced using an automated DNA synthesizer (E.g. Applied Biosystems ABI™ 3900 High-Throughput DNA Synthesizer (Foster City, Calif. 94404 U.S.A.)). Using the same approach, the complement oligonucleotides are synthesized and annealed with the complementary partners to form double stranded oligonucleotides. Pairs of double stranded oligonucleotides (i.e. those that encode adjacent sequences) and joined by ligation to form a larger fragment. These larger fragments are purified by agarose gel electrophoresis and isolated using a gel purification kit (E.g. The QIAEX® II Gel Extraction System, from Qiagen, Santa Cruz, Calif., Cat. No. 12385). This procedure is repeated until the full-length DNA molecule is created. After each round of ligation the fragments can be amplified by PCR to increase the yield. Procedures for de novo DNA synthesis are well known to the art and are described elsewhere (Andre et al., supra, (1998); et al., Haas supra, (1996)); alternatively synthetic genes can be purchased commercially, e.g. from the Midland Certified Reagent Co. (Midland, Tex.).

Following completion of the de novo gene synthesis the integrity of the coding sequence in the resultant DNA fragment is verified by automated dideoxynucleic acid sequencing using an Applied Biosystems Automated DNA Sequencer or using a commercial facility that has the appropriate capabilities and equipment, such as the Biopolymer Core Facility, University of Maryland, Baltimore Md.

VIII. Purification of DNA Vaccine:

The specific method used to purify the DNA vaccines of the present invention is not critical thereto and may be selected from previously described procedures used to purify conventional DNA vaccines (e.g. endotoxin-free large-scale DNA purification kits from Qiagen, Santa Clarita, Calif.; “EndoFree Plasmid Maxi Kit”, cat # 12362), or two rounds of purification using Cesium chloride density gradients (Ausubel, et al., sump (1990)). Alternatively, purified lots of DNA vaccines that co-express an antigen and an adjuvant can be obtained from commercial sources that have the capacity to produce endotoxin-free plasmid DNA preparations using the Good Manufacturing Procedures as outlined by the US Food and Drug Administration, Bethesda Md. Endotoxin levels, which are preferably less than 10 Endotoxin Units (i.e. EU) per ml, are measured using one or more of the well-known procedures (E.g. The Limulus Amebocyte Lysate assay (Cape Cod Associates, Cape Cod, Me.; Cat. No. 3P9702); the chicken embryo toxicity assay (Kotani et al., Infect. Immun., 49:225 (1985)); the rabbit pyrogenicity assay (Kotani et al., supmr (1985)) and the Schwartzman assay (Kotani et al., sums (1985)).

IX. Formulation of a DNA Vaccine that Directs the Coincident Expression of a Vaccine Antigen and a mART:

The specific method used to formulate the novel DNA vaccines described herein is not critical to the present invention and can be selected from previously described procedures used to formulate DNA vaccines, such as formulations that combine DNA vaccine with a physiological buffer (Felgner et al., U.S. Pat. No. 5589466 (1996)); aluminum phosphate or aluminum hydroxyphosphate (e.g. Ulmer et al., Vaccine, 18:18 (2000)), monophosphoryl-lipid A (also referred to as MPL or MPLA; Schneerson et al. J. Immunol., 147: 2136-2140 (1991); e.g. Sasaki et al. Inf. Immunol., 65: 3520-3528 (1997); Lodmell et al. Vaccine, 18: 1059-1066 (2000)), QS-21 saponin (e.g. Sasaki, et al., J. Virol., 72:4931 (1998); dexamethasone (e.g. Malone, et al., J. Biol. Chem. 269:29903 (1994); CpG DNA sequences (Davis et al., J. Immunol., 15:870 (1998); lipopolysaccharide (LPS) antagonist (e.g. Hone et al., U.S. Pat. No. 6,368,604 (1997)), an additional plasmid encoding a cytokine (e.g. Hayashi et al. Vaccine, 18: 3097-3105 (2000); Sin et al. J. Immunol., 162: 2912-2921 (1999); Gabaglia et al. J. Immunol., 162: 753-760 (1999); Kim et al., Eur J Immunol., 28:1089 (1998); Kim et al., Eur. J. Immunol., 28:1089 (1998); Barouch et al., J. Immunol., 161:1875 (1998); Okada et al., J. Immunol., 159:3638 (1997); Kim et al., J. Virol., 74:3427 (2000)), and/or an additional plasmid encoding a chemokine (e.g. Boyer et al., Vaccine 17(Suppl 2):S53 (1999); Xin et al., Clin. Immunol., 92:90 (1999)).

X. Vaccination Strategies:

The DNA vaccine that directs the coincident expression of an antigen and a mART can be introduced into the animal by intravenous, intramuscular, intradermal, intraperitoneally, intranasal and oral inoculation routes. The specific method used to introduce the DNA vaccines that co-express an antigen and a mART described herein into the target animal is not critical to the present invention and can be selected from methods well know in the art for such intramuscular, intravenous, intradermal, intraperitoneally, and intranasal administration of said vaccines (an extensive database of publications describing the above cited vaccination procedures is located at URL: http://www.DNAvaccine.com/Biblio/articles.html).

Oral inoculation of the target animal with the DNA vaccines that direct coincident expression of an antigen and a mART of the present invention can be achieved using a non-pathogenic or attenuated bacterial DNA vaccine vector (Powell et al., U.S. Pat. No. 5,877,159 (1999); Powell et al., U.S. Pat. No. 6,150,170). The amount of the bacterial DNA vaccine vector of the present invention to be administered will vary depending on the species of the subject, as well as the disease or condition that is being treated. Generally, the dosage employed may be about 10³ to 10¹¹ viable organisms, preferably about 10³ to 10⁹ viable organisms, as described (Shata et al., Vaccine 20-621-629 (2001); Shata and Hone, J. Virol. 75-9665-9670 (2001)).

The bacterial DNA vaccine vector carrying the DNA vaccine of the present invention is generally administered along with a pharmaceutically acceptable carrier or diluent. The particular pharmaceutically acceptable carrier or diluent employed is not critical to the present invention. Examples of diluents include a phosphate buffered saline, buffer for buffering against gastric acid in the stomach, such as citrate buffer (pH 7.0) containing sucrose, bicarbonate buffer (pH 7.0) alone (Levine et al, J. Clin. Invest., 19:888-902 (1987); and Black et al J. Infect. Dis., 155:1260-1265 (1987)), or bicarbonate buffer (pH 7.0) containing ascorbic acid, lactose, and optionally aspartame (Levine et al, Lancet, II:467-470 (1988)). Examples of carriers include proteins, e.g., as found in skim milk, sugars, e.g., sucrose, or polyvinylpyrrolidone. Typically these carriers would be used at a concentration of about 0.1-90% (w/v) but preferably at a range of 1 - 10% (w/v).

### EXAMPLE 1

### Recombinant DNA Procedures

i) Reagents, Bacterial Strains and Plasmids

Restriction endonucleases (New England Biolabs Beverly, Mass.), T4 DNA ligase (New England Biolabs, Beverly, Mass.) and Taq polymerase (Life technologies, Gaithersburg, Md.) were used according to the manufacturers' protocols; Plasmid DNA was prepared using small-scale (Qiagen Miniprep^{R} kit, Santa Clarita, Calif.) or large-scale (Qiagen Maxiprep^{R} kit, Santa Clarita, Calif.) plasmids DNA purification kits according to the manufacturer's protocols (Qiagen, Santa Clarita, Calif.); Nuclease-free, molecular biology grade milli-Q water, Tris-HCl (pH 7.5), EDTA pH 8.0, 1M MgCl₂, 100% (v/v) ethanol, ultra-pure agarose, and agarose gel electrophoresis buffer were purchased from Life technologies, Gaithersburg, Md. DNA ligation reactions and agarose gel electrophoresis were conducted according to well-known procedures (Sambrook, et al., supra (1989); (Ausubel, et al., supra (1990)).

PCR primers were purchased from the University of Maryland Biopolymer Facility (Baltimore, Md.) and were synthesized using an Applied Biosystems DNA synthesizer (model 373A). PCR primers were used at a concentration of 200 μM and annealing temperatures for the PCR reactions were determined using Clone manager software version 4.1 (Scientific and Educational Software Inc., Durhan N.C.). PCRs were conducted in a Strategene Robocycler, model 400880 (Strategene, La Jolla, Calif.). Annealing, elongation and denaturation times in the PCRs were set according to well-known procedures.

Nucleotide sequencing to verify the DNA sequence of each recombinant plasmid described in the following examples was accomplished by conventional automated DNA sequencing techniques using an Applied Biosystems automated sequencer, model 373A.

Escherichia coli strain Sable2^{R} was purchased from Life Technologies (Bethesda, Md.) and served as host of the recombinant plasmids described in the examples below.

Plasmid pCVD002 (Lochman and Kaper, J. Biol. Chem., 258:13722 (1983)) served as a source of the CtxA I-encoding sequences (kindly provided by Dr. Jim Kaper, Department of Microbiology and Immunology, University of Maryland, Baltimore).

Recombinant plasmids were introduced into E. coli strain Stable2^{R} by electroporation using a Gene Pulser (BioRad Laboratories, Hercules, Calif.) set at 200 Ω, 25 μF and 2.5 kV as described (Hone, et al., Vaccine, 9:810 (1991)).

Bacterial strains were grown on tryptic soy agar (Difco, Detroit Mich.) or in tryptic soy broth (Difco, Detroit Mich.), which were made according to the manufacturer's directions. Unless stated otherwise, all bacteria were grown at 370C. When appropriate, the media were supplemented with 100 μg/ml ampicillin (Sigma, St. Louis, MO).

Bacterial strains were stored at −80° C. suspended in tryptic soy broth containing 30% (v/v) glycerol at ca. 10⁹ colony-forming units (herein referred to as “cfu”) per ml.

Plasmid pCITE4a, which contains the IRES of equine encephalitis virus, was purchased from Novagen (Madison Wis.).

Plasmid pcDNA3.1_{ZEO}, which contains the colE1 replicon, an ampicillin-resistance allele, the CMV immediate-early promoter, a multicloning site and the bovine hemoglobin poly-adenosine sequence, was purchased from Clonetech (Palo Alto, Calif.).

Plasmid pEF1a-syngp120MN carrying synthetic DNA encoding HIV-1_{MN} gp120 (referred to herein as hgp120), in which the native HIV-1 leader peptide was replaced by the human CD5 leader peptide and the codons are optimized for expression in mammalian cells is described elsewhere (Andre et al., supra, (1998); et al., Haas supra, (1996)).

Restriction endonuclease digestion, ligation, and plasmid DNA preparation techniques were all conducted as described earlier. Nucleotide sequencing to verify the structure of each recombinant plasmid described in the following examples was accomplished by standard automated sequencing techniques (Applied Biosystems automated sequencer, model 373A).

### EXAMPLE 2

### Vaccination and Immunological Procedures

Source of Laboratory Animals and Handling:

BALB/c and C57BV/6 mice aged 6-8 weeks were obtained from Charles River (Bar Harbor, Me.). All of the mice were certified specific-pathogen free and upon arrival at the University of Maryland Biotechnology Institute Animal Facility were maintained in a microisolator environment and allowed to fee and drink ad lib.

Vaccination Procedures:

Groups of 6 mice were vaccinated intramuscularly with 1 - 100 pg of endotoxin-free (<0.5 EU per mg of DNA) plasmid DNA suspended in saline (0.85% (w/v) NaCl), as described (Felgner et al., U.S. Pat. No. 5,589,466 (1996)). Booster vaccinations were given using the same formulation, route and dose as used to prime the mice; the spacing of the doses is outlined below.

Serum Enzyme-Linked Immunosorbent Assays (ELISAs):

Blood (ca. 100 μl per mouse) was collected before and at weekly intervals after vaccination. The presence of gp120-specific IgG in pooled sera collected from the vaccinated mice was determined by ELISA. Aliquots (0.3 μg suspended in 100 μl PBS, pH 7.3) of purified glycosylated HIV-IMN gp120 (Virostat, Portland) were added to individual wells of 96-well Immulon plates (Dynex technologies Inc, Virginia, USA). After incubating 16-20 hr at 4° C., the plates were washed three times with washing buffer (Kirkegaard and Perry Laboratories, Gaithersburg, Md.) and 200 μl of blocking buffer (Kirkegaard and Perry Laboratories, Gaithersburg, Md., USA) was added and the plates were incubated for 1 hr at 25° C. After the blocking was complete, duplicated sets of each serum sample were diluted serially in 3-fold increments (Starting at 1:10) in blocking buffer and incubated for 1 hr at room temperature. Then, the plates were washed six times with washing buffer and 100 μl of horseradish peroxidase-labelled goat anti-mouse IgG (Sigma Immunochemicals, USA), diluted in 1/2000 in blocking buffer, was added to each well and the plates were incubated for 1 hr at 25° C. The plates were washed an additional six times with washing buffer and 100 μl of ABTS substrate (Kirkegaard and Perry Laboratories, Gaithersburg, Md., USA) was added and the plates were incubated for 30 min at 25° C. The absorbance was measured at 405 nm using a Wallac Dynamic Reader, model 1420 (Turku, Finland). A similar procedure was conducted to measure gp120-specific IgG subtypes, IgG1, IgG2a and IgG2b, except that rat anti-mouse IgG1, IgG2a, and IgG2b antibodies conjugated to horseradish peroxidase (diluted 1:8000, 1:2000 and 1:1000, respectively; BioSource International, Keystone, USA) were using in place of the goat anti-mouse IgG.

### EXAMPLE 3

Connstniction of DNA Vaccines Encoding a Viral Antigen and a mART:

In this example a novel DNA vaccine was constructed, herein designated pOGL1-A1-S63K, which co-expresses an antigen (i.e. gp120 of HIV-1_{MN}) and a mutant derivative of the Al domain of the A subunit of Ctx (referred to herein as “CtxA1”) that harbors a lysine substitution at amino acid no. 63 (i.e. herein referred to as “CtxA1-S63K”) in place of the serine that is present in the parental CtxA1.

i) Expression vector pcDNA3.1_{ZEO} was purchased from Invitrogen (Carlsbad, Calif.) and carries the CMV promoter that is active in a wide spectrum of eukaryotic cells.

ii) Construction of DNA vaccine pOGL1 was achieved by PCR-amplifying hgp120 from a plasmid pEF1α-syngp120_{MN} (Andre et al., supra, (1998); et al., Haas supra, (1996)) using forward primer 5′-GGGGGGGGATCCATGCCCATGGGGTCTCTGCAACCGCTG (SEQ ID #1) and reverse primer 5′-GGGGGCGGCCGCTTATTAGGCGCGCTTCTCGCGCTGCACCACGCG (SEQ ID #2) using the PCR procedure outlined in example 1 above. The resultant PCR-generated DNA fragment was digested with restriction endonucleases BamHI and NotI and annealed (E.g. by ligation with T4 ligase) with BamHI- and NotI-digested pcDNA3.1_{ZEO} DNA (Invitrogen, Carlsbad, Calif., Cat. No. V860-20). The ligated DNA was introduced into E. coli strain Stable2^{R} (Life Technologies, Gaithersburg, Md.) by electroporation. Plasmid DNA was prepared from 2 ml liquid cultures of individual clones and used to screen for a clone that carried a plasmid with the appropriate restriction endonuclease digestion pattern. One such clone, referred to herein as “H1058”, containing the desired plasmid (referred to herein as “pOGL1”), which is pcDNA3.1_{ZEO} containing the BamHI-NotI hgp120 fragment, was stored at −80° C. Additional analysis by restriction endonuclease digestion, PCR of the hgp120 DNA, and dideoxynucleotide sequencing of the cloned hgp120 DNA in pOGL1 was conducted to verify that the hgp120 DNA was not altered during construction.

iii) DNA encoding the IRES of equine encephalitis virus, herein referred to as the cap-independent translational enhancer (U.S. Pat. No. 4,937,190, which is herein incorporated by reference), was amplified from plasmid pCITE4a (Novagen, Madison Wis.; Cat. No. 69912-1; U.S. Pat. No. 4,937,190) using forward primer 5′-ATAAGAATGCGGCCGCTAAGTAAGTAACTTAAGTTCCGGTTATTTTCCACGATATTGCCGTCTT TTGGCAA (SEQ ID #3) and reverse primer 5′-GCCAAATACATGGCCATATTATCATCGTGTTTTTCAAAGGAA (SEQ ID #4).

iv) DNA encoding CtxA1-S63K was amplified from plasmid pOGL1-A1 [13], which has a copy of CtxA1. The nucleotide sequence of ctxA1-S63K was obtained from (SEQ

Nucleotide sequence of CtxA1-S63K

| | |
|---|---|
| 1 | AATGATGATA AGTTATATCG GGCAGATTCT AGACCTCCTG ATGAAATAAA GCAGTCAGGT |
| 61 | GGTCTTATGC CAAGAGGACA GAGTGAGTAC TTTGACCGAG GTACTCAAAT GAATATCAAC |
| 121 | CTTTATGATC ATGCAAGAGG AACTCAGACG GGATTTGTTA GGCACGATGA TGGATATGTT |
| 181 | TCCACCAAAA TTAGTTTGAG AAGTGCCCAC TTAGTGGGTC AAACTATATT GTCTGGTCAT |
| 241 | TCTACTTATT ATATATATGT TATAGCCACT GCACCCAACA TGTTTAACGT TAATGATGTA |
| 301 | TTAGGGGCAT ACAGTCCTCA TCCAGATGAA CAAGAAGTTT CTGCTTTAGG TGGGATTCCA |
| 361 | TACTCCCAAA TATATGGATG GTATCGAGTT CATTTTGGGG TGCTTGATGA ACAATTACAT |
| 421 | CGTAATAGGG GCTACAGAGA TAGATATTAC AGTAACTTAG ATATTGCTCC AGCAGCAGAT |
| 481 | GGTTATGGAT TGGCAGGTTT CCCTCCGGAG CATAGAGCTT GGAGGGAAGA GCCGTGGATT |
| 541 | CATCATGCAC CGCCGGGTTG TGGGAATGCT CCAAGATCAT CG_{END} |

ID #5) GenBank (Accession # A16422) and modified by replacing the serine-63 TCA codon (nucleotides 187-189; See sequence above) with a lysine codon (i.e. AAA). The mutant derivative of CtxA1, CtxA1-S63K, was generated using the QuikChange® Site-Directed Mutagenesis Kit (Catalog #200518, Stratagene). The site-directed mutagenesis process entailed whole-plasmid PCR using pOGL1-A1 DNA as template, forward primer 5′-TGTTTCCCACCAAAATTAGTTTGAGAAGTGC (SEQ ID # 6) and reverse primer 5′-CAAACTAATTTTGGTGGAAACATATCCATC (SEQ ID #7); this procedure modified nucleotides 187-189 by replacing TCA (i.e. serine-63 codon) with a lysine codon (i.e. 5′-AAA). The resultant PCR-generated plasmid was digested with DpnI to remove the template DNA and the digested DNA was introduced into E. coli Stable2® by chemical transformation. The transformed bacilli were cultured on tryptic soy agar (Difco, Detroit Mich.) supplemented with 100-μg/ml ampicillin at 30° C. for 16 hr.

Isolated colonies were selected and grown overnight in 3 ml of LB medium supplemented with 100 μg/ml ampicillin. DNA was extracted from overnight liquid cultures using a Qiagen mini plasmid DNA preparation kit (Cat No Q7106). Plasmid PCR using primers specific for ctxA1-S63K, and agarose gel electrophoresis were conducted to screen for an appropriate derivative; several isolates tested positive for ctxA1-S63K insert and strain containing the appropriate plasmid (herein referred to as “pOGL1-A1-S63K”) were stored at −80° C. as described above. One such isolate was used as the source of pOGL1-A1-S63K DNA for the vaccination studies below.

### EXAMPLE 4

Construction of DNA Vaccines Encoding a Bacterial Antigen and a mART:

In this example a novel DNA vaccine was constructed, herein designated pCtxA1-E29H, which co-expresses an antigen (i.e. the receptor-binding domain of protective antigen of Bacillus anthracis (Price, et al. Infect. Immun. 69, 4509-451 (2001)) and a mutant derivative of the A1 domain of the A subunit of Ctx (referred to herein as “CtxA1”) that harbors a histidine substitution at amino acid no. 29 (i.e. herein referred to as “CtxA1 -E29H”) in place of the glutamine that is present in the parental CtxA1.

i) Expression vector pcDNA3.1_{ZEO} can be purchased from Invitrogen (Carlsbad, Calif.) and carries the CMV promoter that is active in a wide spectrum of eukaryotic cells.

ii) The DNA sequence encoding a truncated derivative of protective antigen (tPA) of B. anthracis is obtained by amplifying a truncated derivative of the pagA gene (Genbank accession no. AF268967) in pCPA ([73]; kindly provided by Dr. Darrell Galloway, Department of Microbiology, Ohio State University, Ohio) using conventional PCR procedures so that BamHI and NotI sites are created at the 5-prime and 3-prime ends, respectively (Example 1). The PCR-generated tPA fragment is digested with BamHI (New England Biolabs) and NotI (New England Biolabs) and inserted, using T4 DNA ligase (New England Biolabs), into BamHI-, NotI-digested pcDNA3.1_{ZEO}. The ligated DNA is introduced into E. coli strain Stable2^{R} (Life Technologies, Gaithersburg, Md.) by electroporation. Plasmid DNA is prepared from 2 ml liquid cultures of individual clones and used to screen for a clone that carried a plasmid with the appropriate restriction endonuclease digestion pattern. Isolates containing the desired plasmid (referred to herein as “pcDNA::tPA”), which is pcDNA3.1^{ZEO} containing the BamHI-NotI tPA fragment, are stored at −80° C. Additional analyses by restriction endonuclease digestion, PCR of the pagA DNA, and dideoxynucleotide sequencing of the cloned pagA in pcDNA::tPA is conducted to verify that the tPA-encoding DNA is not altered during construction.

iii) DNA encoding CtxA1-E29H was amplified from plasmid pRc/CMV-A1 (Bagley et al. Vaccine. 21:3335-3341 (2003)), which has a copy of wild-type CtxA1. The (SEQ

Nucleotide sequence of CtxA1-E29H

| | |
|---|---|
| 1 | AATGATGATA AGTTATATCG GGCAGATTCT AGACCTCCTG ATGAAATAAA GCAGTCAGGT |
| 61 | GGTCTTATGC CAAGAGGACA GAGTCACTAC TTTGACCGAG GTACTCAAAT GAATATCAAC |
| 121 | CTTTATGATC ATGCAAGAGG AACTCAGACG GGATTTGTTA GGCACGATGA TGGATATGTT |
| 181 | TCCACCTCAA TTAGTTTGAG AAGTGCCCAC TTAGTGGGTC AAACTATATT GTCTGGTCAT |
| 241 | TCTACTTATT ATATATATGT TATAGCCACT GCACCCAACA TGTTTAACGT TAATGATGTA |
| 301 | TTAGGGGCAT ACAGTCCTCA TCCAGATGAA CAAGAAGTTT CTGCTTTAGG TGGGATTCCA |
| 361 | TACTCCCAAA TATATGGATG GTATCGAGTT CATTTTGGGG TGCTTGATGA ACAATTACAT |
| 421 | CGTAATAGGG GCTACAGAGA TAGATATTAC AGTAACTTAG ATATTGCTCC AGCAGCAGAT |
| 481 | GGTTATGGAT TGGCAGGTTT CCCTCCGGAG CATAGAGCTT GGAGGGAAGA GCCGTGGATT |
| 541 | CATCATGCAC CGCCGGGTTG TGGGAATGCT CCAAGATCAT CG_{END} |

ID # 8) nucleotide sequence of ctxA1-E29H is obtained from GenBank (Accession # A16422) and modified by replacing the glutamine-29 GAG codon (nucleotides 85-87; See sequence above) with a histidine codon (i.e. CAC). The mutant derivative of CtxA1, CtxA1-E29H, can be generated using the QuikChange® Site-Directed Mutagenesis Kit (Catalog #200518, Stratagene). The site-directed mutagenesis process entailed whole-plasmid PCR using pCTA-A1 DNA as template, forward primer 5′-CAAGAGGACAGAGTCACTACTTTGACCGAG (SEQ ID # 9) and reverse primer 5′-GTTCTCCTGTCTCAGTGATGAAACTGGCAC (SEQ ID #10); this procedure modified nucleotides 187-189 by replacing GAG (i.e. glutamine-29 codon) with a histidine codon (i.e. 5′-CAC). The resultant PCR-generated plasmid is digested with DpnI to remove the template DNA and the digested DNA is introduced into E. coli Stable2® by chemical transformation. The transformed bacilli are cultured on tryptic soy agar (Difco, Detroit Mich.) supplemented with 100-μg/ml ampicillin at 30° C. for 16 hr.

Isolated colonies are selected and grown overnight in 3 ml of LB medium supplemented with 100 μg/ml ampicillin. DNA is extracted from overnight liquid cultures using a Qiagen mini plasmid DNA preparation kit (Cat No Q7106). Plasmid PCR using primers specific for ctxA1-E29H, dideoxysequencing and agarose gel electrophoresis are conducted to screen for an appropriate derivative; isolates that test positive for ctxA1-E29H insert and strain containing the appropriate plasmid (herein referred to as “pRc/CMV::A1-E29H”) are stored at −80° C. as described above.

### EXAMPLE 5

Immunogenicity of a DNA Vaccine that Directs the Coincident Expression of gp120 and a mART:

The adjuvant activity of CtXA1-S63K in DNA vaccine pOGL1-A1-S63K (Example 3) was characterized by comparing the immunogenicity of DNA vaccine pOGL1 that expresses gp120 alone, to that of bicistronic DNA vaccine pOGL1-A1-S63K that expresses both gp120 and CtxA1-S63K) in BALB/c mice. Accordingly, groups of 3 BALB/c mice were vaccinated intramuscularly with three 40 μg-doses of endotoxin-free plasmid DNA on days 0, 14 and 42. A negative control group of 3 BALB/c mice received three dose 40 μg-doses of plasmid pcDNA3.1 DNA using the same protocol and intervals between doses.

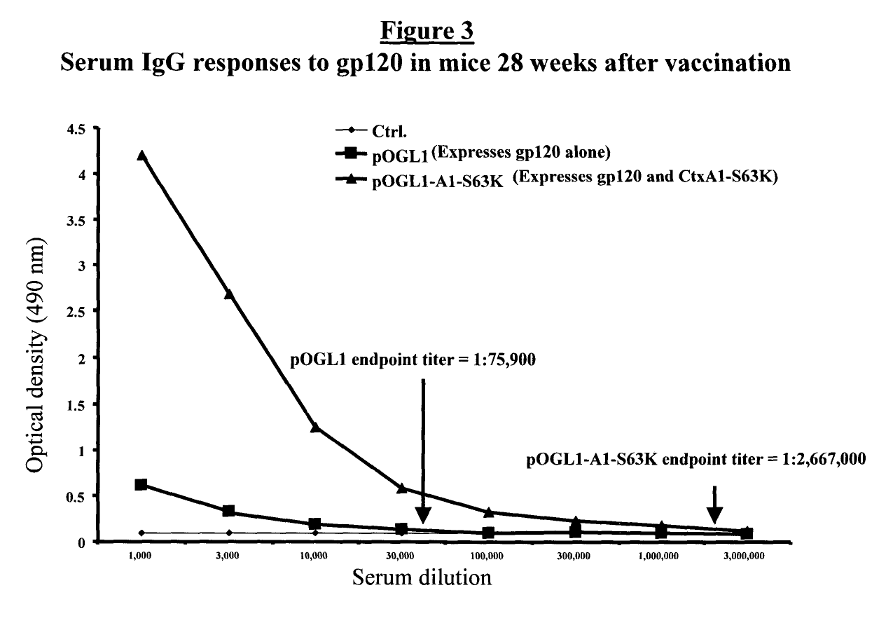

Sera were collected before and at regular intervals after vaccination, and used to measure the serum IgG response against HIV-1MN gp120 by ELISA (Example 2). This experiment demonstrates that mice vaccinated with bicistronic DNA vaccine pOGL1-A1-S63K developed a serum IgG response against gp120 that was significantly greater and remained elevated longer than the analogous serum IgG response in mice vaccinated with the DNA vaccine that expressed gp120 alone (i.e. pOGL1; FIG. 3).

### EXAMPLE 6

### Interpretation of the Results

The above unanticipated finding indicates that in contrast to the mutant CT-S63K holotoxin (i.e the protein form) which displays little adjuvant activity, DNA vaccines which express mARTs that are devoid of ADP-ribosyltransferase activity (i.e. CtxA1-S63K) retain potent adjuvanticity.

LT-S63K and CT-S63K holotoxins are poor adjuvants. Pertussis toxin and the adenylate cyclase toxin from Bordetella pertussis activate human monocyte-derived dendritic cells and dominantly inhibit cytokine production through a cAMP dependent pathway (J. Leukoc. Biol. 2002 Nov.; 72(5):962-9). Progress toward the development of a bacterial vaccine vector that induces high titer long lived broadly neutralizing antibodies against HIV-1 can be found in Fouts et al., FEMS Immunol. Med. Microbiol. 2003 Jul. 15; 37(2-3):129-34. Mucosal adjuvanticity and immunogenicity of LTR72, a novel mutant of E.coli heat labile enterotoxin with partial knockout of ADP-ribosyltransferase activity is discussed in Giuliani et al., J. Exp. Med. 1998 Apr 6; 187(7): 1123-32.

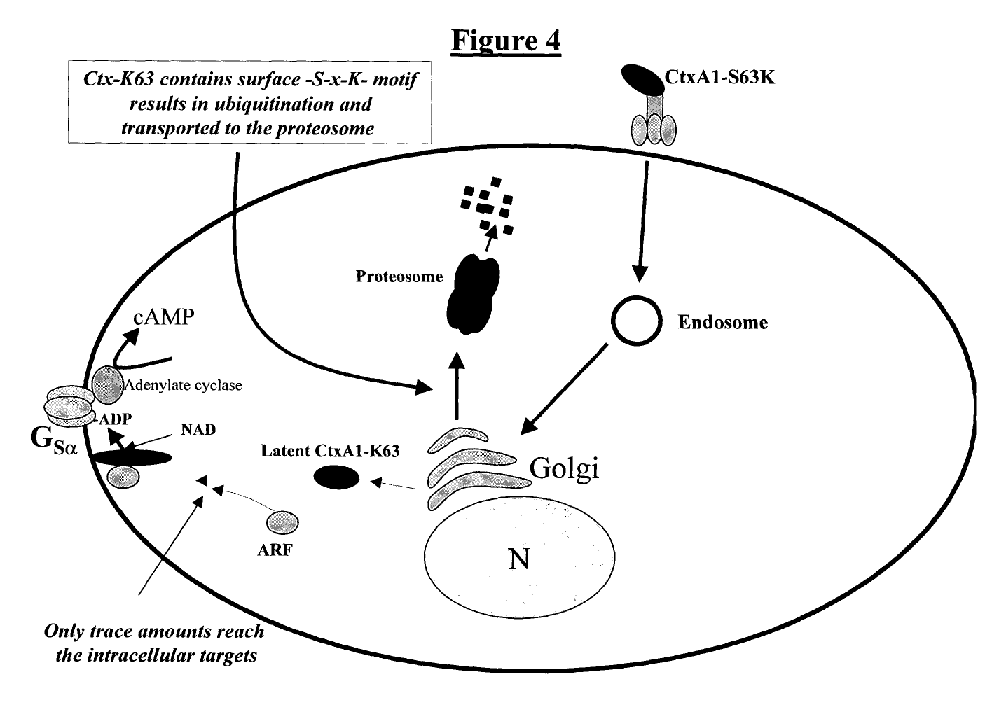

The basis for this difference between DNA vaccines which express mARTs and the protein form is likely due to differences in the intracellular trafficking pathways employed by the purified holotoxin, compared to CtxA1-S63K when expressed by a DNA vaccine. The mutant CT-S63K holotoxin when added in the form of a purified protein must traffic via the golgi apparatus to reach the cell cytoplasm and during this transport is exposed to the cellular ubiquitination/proteosome degredation machinery (FIG. 4). The presence of the surface-exposed lysine (i.e. S63K) serves as a cognate recognition motif for ubiquitination and proteosome degradation, substantially preventing interaction between the A1-S63K subunit of the mutant holotoxin with the host ADP-Ribosyltransferase Factor (herein referred to as ARF), and the subsequent ADP-ribosylation of Gsa and activation of adenylate cyclase (FIG. 4). The reduced ability to reach the host ARF explains may why mutants of cholera toxin mutants that carry amino acid substitutions that are recognized by the host ubiquitination and proteosome degradation apparatus (e.g CT-S63K or LT-S63K) display relatively insipient adjuvant activity, relative to wild-type CT or LT [14].

Furthermore, the interaction between CtxA1 (or LtxA1) and ARF augments the ADP-ribosyltransferase catalytic activity and substantially increases intracellular levels of cyclic-adenosine monophosphate (herein referred to as “cAMP”) in treated cells. Thus, this interaction is required for maximal ADP-ribosyltransferase activity of wild-type CT (Jobling, et al., Proc. Natl. Acad. Sci. U S A, 97-14662-14667 (2000)), and the ability to increase cAMP levels is directly linked to the toxicity of ADP-ribosyltransferase toxins [14].

In summary, the poor adjuvant properties of purified mutant ADP-ribosyltransferase holotoxins that are incapable of binding NAD (such as CT-S63K) was probably due to ubiquitination of such molecules in the golgi and accelerated proteosome degradation.

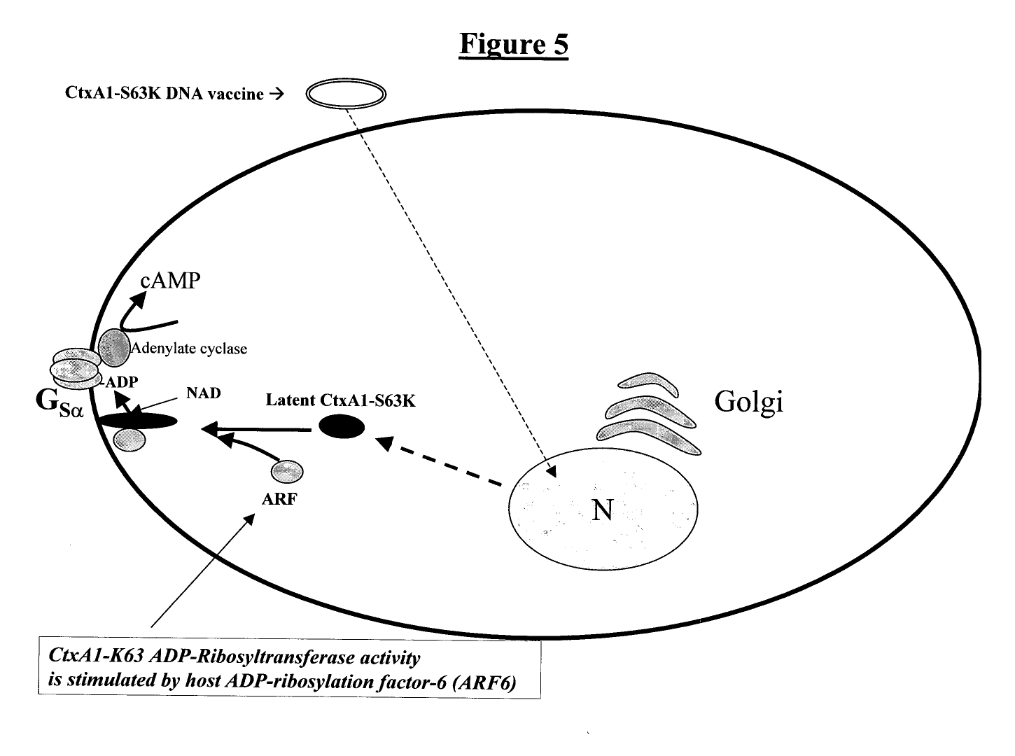

In contrast, delivery of mARTs by in the DNA vaccine mode bypasses the golgi apparatus, thereby avoiding ubiquitination and proteosome degradation, and allowing access to the endogenous host ARFs (FIG. 5). Some mARTs (such as CT-S63K) are incapable of binding NAD and thus retain the capacity to access endogenous ARFs. Furthermore, it is known that mARTs (such as CT-S63K) that are incapable of binding NAD retain the ability to interact with endogenous ARFs (Stevens, et al., Infect Immun. 67:259-265 (1999)). The role of this interaction per se in the adjuvant properties of ADP-ribosyltransferase toxins, however, has not heretofore been evaluated.

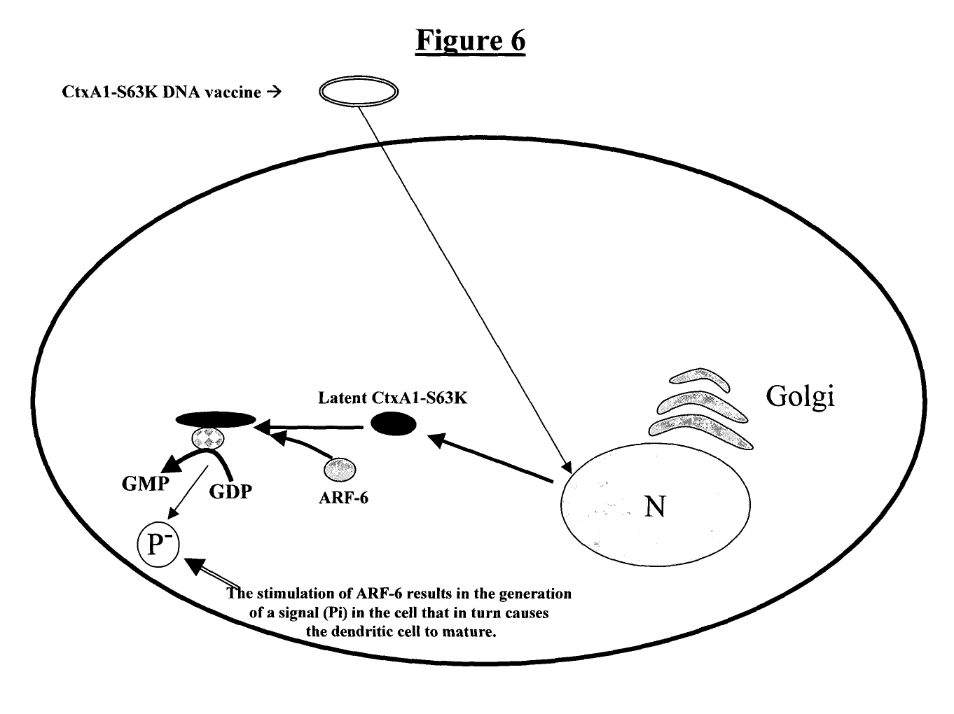

Thus, example 5 presents a novel and unexpected finding that delivery of a mART which is incapable of binding NAD (e.g. CT-S63K) to the appropriate cellular compartment results in significant adjuvant activity. Presumably expression CtxA1-S63K by the DNA vaccine in dendritic cells (key antigen presenting cell involved in promoting DNA vaccine-induced immune responses [16-18]) causes said cells to differentiate into a mature antigen presenting cells, thereby augmenting the immunogenicity of an immunogen that is coincidently expressed with said mART. One mechanism through which CtxA1-S63K DNA vaccine retains adjuvant activity may a conformational change following the interaction between CtxA1-S63K and the host ARF-6, thereby opening the GTP-binding cleft in ARF-6. Thus, the interaction between of CtxA1 to ARF may invoke the GTPase activity of ARF-6 resulting in increased membrane recycling and maturation of dendritic cells that harbor said DNA vaccine into a mature antigen presenting cell, which in turn promote the robust immune responses to the DNA vaccine-encoded antigen (FIG. 6).

### EXAMPLE 7

### Advantages of mARTs

A key advantage possessed by DNA vaccines that express a mART is that such vaccines are likely to have a broader safety profile in large population studies. In addition, the growth of strains harboring DNA vaccines that express a mART have proven to be more stable and capable of growing the greater optical densities. Thus, strains harboring said mutant DNA vaccine produce about 4-fold more viable bacilli per ml of culture (i.e. for 16 hr at 37° C. in LB with agitation), compared to parallel cultures of strains that carrying a DNA vaccine that expresses a wild-type ADP-ribosyltransferase toxin. This finding has obvious manufacturing implications and bodes well for the application of this technology to large-scale public health vaccination programs.

### REFERENCES:

1 Hone, D., Morona, R., Attridge, S. & Hackett, J. Construction of defined galE mutants of Salmonella for use as vaccines. J. Infect. Dis. 1987, 156(1), 167-174.

2 Hone, D., Attridge, S., van den Bosch, L. & Hackett, J. A chromosomal integration system for stabilization of heterologous genes in Salmonella-based vaccine strains. Microbial. Path. 1988, 5(6), 407418.

3 Hone, D. M., Harris, A. M., Chatfield, S., Dougan, G. & Levine, M. M. Construction of genetically defined double aro mutants of Salmonella typhi. Vaccine 1991, 9(11), 810-816.

4 Hone, D. M., Harris, A. M., Lim, V. & Levine, M. M. Construction and characterization of isogenic O-antigen variants of Salmonella typhi. Mol. Microbiol. 1994, 13(3), 525-530.

5 Noriega, F. R., Wang, J. Y., Losonsky, G., Maneval, D. R., Hone, D. M. & Levine, M. M. Construction and characterization of attenuated delta aroA delta virG Shigella flexneri 2a strain CVD 1203, a prototype live oral vaccine. Infect. Immun. 1994, 62(11), 5168-5172.

6 Fouts, T. R., Tuskan, R. G., Chada, S., Hone, D. M. & Lewis, G. K. Construction and immunogenicity of Salmonella typhimurium vaccine vectors that express HIV-1 gp120. Vaccine 1995, 13(17), 1697-1705.

7 Fouts, T. R., Lewis, G. K. & Hone, D. M. Construction and characterization of a Salmonella typhi-based human immunodeficiency virus type 1 vector vaccine. Vaccine 1995, 13(6), 561-569.

8 Wu, S., Pascual, D. W., VanCott, J. L. et al. Immune responses to novel Escherichia coli and Salmonella typhimurium vectors that express colonization factor antigen I (CFA/I) of enterotoxigenic E. coli in the absence of the CFA/I positive regulator cfaR. Infect. Immun. 1995, 63(12), 4933-4938.

9 Hone, D. M., Wu, S., Powell, R. J. et al. Optimization of live oral Salmonella-HIV-1 vaccine vectors for the induction of HIV-specific mucosal and systemic immune responses. J. Biotech. 1996, 44(1-3), 203-207.

10 Powell, R. J., Lewis, G. K. & Hone, D. M. Introduction of eukaryotic expression cassettes into animal cells using a bacterial vector delivery system. In: Vaccine96. Molecular Approaches to the Control of Infectious Disease. Brown, F., Norrby, E., Burton, D. and Mekalanos, J. (Eds). Cold Spring Harbor Press, New York, N.Y. 1996, Pp183-187.

11 Wu, S., Pascual, D. W., Lewis, G. K. & Hone, D. M. Induction of mucosal and systemic responses against human immunodeficiency virus type 1 glycoprotein 120 in mice after oral immunization with a single dose of a Salmonella-HIV vector. AIDS Res. Hum. Retrovir. 1997, 13(14), 1187-1194.

12 Ansorge, W., Sproat, B., Stegemann, J., Schwager, C. & Zenke, M. Automated DNA sequencing: ultrasensitive detection of fluorescent bands during electrophoresis. Nucleic Acids Res 1987, 15(11), 4593-4602.

13 Bagley, K. C., Fouts, T. R., Carbonetti, N., DeVico, A. L., Lewis, G. K. & Hone, D. M. Inimunogenicity of a dicistronic DNA vaccine that directs coincident expression of the 120 kDa glycoprotein of human immunodeficiency virus and the catalytic domain of cholera toxin. (Submitted). 2002.

14 Fouts, T. R., DeVico, A. L., Onyabe, D. Y. et al. Progress toward the development of a bacterial vaccine vector that induces high-titer long-Lived broadly neutralizing antibodies against HIV- 1. FEMS 2002, (In press).

15 Giuliani, M. M., Del Giudice, G., Giannelli, V. et al. Mucosal adjuvanticity and immunogenicity of LTR72, a novel mutant of Escherichia coli heat-labile enterotoxin with partial knockout of ADP-ribosyltransferase activity. J. Exp. Med. 1998, 187(7), 1123-1132.

16 Porgador, A., Irvine, K. R., Iwasaki, A., Barber, B. H., Restifo, N. P. & Germain, R. N. Predominant role for directly transfected dendritic cells in antigen presentation to CD8+T cells after gene gun immunization. J. Exp. Med. 1998, 188(6), 1075-1082.

17 Casares, S., Inaba, K., Brumeanu, T. D., Steinman, R. M. & Bona, C. A. Antigen presentation by dendritic cells after immunization with DNA encoding a major histocompatibility complex class 11-restricted viral epitope. J. Exp. Med. 1997, 186(9), 1481-1486.

18 You, Z., Huang, X., Hester, J., Toh, H. C. & Chen, S. Y. Targeting dendritic cells to enhance DNA vaccine potency. Cancer Res. 2001, 61(9), 3704-3711.

While the invention has been described in detail, and with reference to specific embodiments thereof, it will be apparent to one of ordinary skill in the art that various changes and modifications can be made therein without departing from the spirit and scope thereof.

I claim:

1. A DNA vaccine, comprising:
at least one genetic sequence encoding a mutant ADP-ribosyltransferase toxin (mART); and
at least one genetic sequence encoding an antigen.

2. The DNA vaccine of claim 1 wherein said mART is derived form a cholera toxin.

3. The DNA vaccine of claim 1 wherein said mART is derived form a pertussis toxin.

4. The DNA vaccine of claim 1 wherein said mART is derived form a heat labile toxin of enterotoxuigenic Escherichia coli.

5. The DNA vaccine of claim 1 wherein said mART includes a mutation selected from the group consisting of R7K, R13H, E29H, H35R, L41F, F50S, S61K, S63K, S63Y, V35D, V97K, Y104K, P106S, H171Y, and combinations thereof.

6. The DNA vaccine of claim 1 further comprising an expression vector, said at least one genetic sequence encoding said mART is associated with said expression vector.

7. The DNA vaccine of claim 6 wherein said at least one genetic sequence encoding said antigen is associated with said expression vector.

8. The DNA vaccine of claim 7 further comprising an internal ribsome entry site positioned between said at least one genetic sequence encoding said mART and said at least one genetic sequence encoding said antigen.

9. The DNA vaccine of claim 6 wherein said expression vector is a eukaryote.

10. The DNA vaccine of claim 9 further comprising at least one eukaryote promoter sequence associated with said expression vector.

11. The DNA vaccine of claim 10 wherein said at least two eukaryote promoter sequence associated with said expression vector.

12. The DNA vaccine of claim 6 wherein said at least one genetic sequence encoding said antigen is associated with a second expression vector that is separate from said expression vector associated with said at least one genetic sequence encoding a mART.

13. The DNA vaccine of claim 1 wherein said antigen is bacterial.

14. The DNA vaccine of claim 1 wherein said antigen is viral.

15. A vaccine composition, comprising:
an antigen; and
at least one genetic sequence encoding a mutant ADP-ribosyltransferase toxin (mART).

16. A vaccine composition, comprising:
a DNA vaccine that expresses an antigen; and
at least one genetic sequence encoding a mutant ADP-ribosyltransferase toxin (mART).

17. A method of vaccinating a patient, comprising:
introducing into said patient at least one genetic sequence encoding a mutant ADP-ribosyltransferase toxin (mART) and at least one genetic sequence encoding an antigen;
expressing said mART and said antigen after said introducing step.

18. The DNA vaccine of claim 1 wherein said step of introducing is accomplished using an expression vector which both said genetic sequence encoding said mART and said genetics sequence encoding said antigen are associated with.

19. The method of claim 18 wherein said expression vector includes at least two eukaryote promoters.

20. The method of claim 18 wherein said expression vector includes an internal ribosome entry site positioned between said at least one genetic sequence encoding said mART and said at least one genetic sequence encoding said antigen.
